(19) United States
(12) Patent Application Publication
(10) Pub. No.: US 2026/0232872 A1
(43) Pub. Date: Aug. 13, 2026

(54) BIODEGRADABLE INTERMITTENT SELF CATHETERISATION CATHETER

(71) Applicant: Gary Francis HUNTER, Exeter Devon (GB)

(72) Inventor: Gary Francis HUNTER, Exeter Devon (GB)

(21) Appl. No.: 19/153,474

(22) PCT Filed: Feb. 2, 2024
(86) PCT No.: PCT/IB2024/050976

§ 371 Date: Aug. 4, 2025

(30) Foreign Application Priority Data
Feb. 6, 2023 (GB) 2301628.0

(51) Int. Cl. A61L 29/04; A61M 25/00

(52) U.S. Cl. CPC A61L 29/045; A61L 29/041; A61L 29/043; A61M 25/002; A61M 2205/02

(57) ABSTRACT

An ISC (intermittent self catheterisation) catheter 10 is formed of a blend of synthetic polymer, e.g. PVC, and a natural biodegradable organic material such as collagen, chitosan or corn starch. The catheter 10 is supplied in a package 1 sealed in an envelope 2 formed of a material which is impermeable to moisture. The catheters are therefore suitable to be disposed of in regular collection schemes, but when sealed within the moisture-excluding envelope 2 the catheters have a normal shelf life.

### TECHNICAL FIELD OF THE INVENTION

This invention relates to catheters for use in intermittent self catheterisation.

### BACKGROUND

Intermittent self catheterisation (ISC) of the bladder is a widely performed procedure, which may, for example, be necessitated by operative procedures or spinal cord injuries where the patient loses the ability to control their bladder. Most ISC catheters are currently made from polyvinyl chloride (PVC), which is inexpensive, strong, soft and flexible and rarely causes adverse reactions in body tissues.

Whilst catheters used by medical professionals are generally sterilised and re-used, for hygienic reasons ISC catheters are essentially single use and are not regarded as recyclable. people who perform ISC may use several catheters in the course of a day, most of which will end up in landfill where, over time, the polymer leaches toxic additives. Furthermore, if used catheters are incinerated they can emit toxic products such as dioxins and heavy metals, hydrogen chloride gas and dioxins.

Photodegradable plastics have been developed which enable synthetic polymers to break down on exposure to light. They are mainly used in agriculture and items which are commonly discarded in the environment such as trays, bottles and packaging materials. The rate of degradation is highly dependent upon exposure time and light intensity. Chemically degradable plastics have also been produced using additives which are classified as oxo-degradants, but they are are known to leave micro-plastics in the environment which can enter water courses and end up in marine environments where they cause serious harm to marine life. They can also enter the food chain and are increasingly being recognised as potentially harmful to humans and animal life in general.

Biodegradable plastics have been developed which can completely decompose in the environment, but although they have good mechanical properties and are stable on exposure to light, they are very expensive in comparison with general use plastics such as PVC, and their suitability for medical use has yet to be assessed.

CN 106188985A discloses a medical catheter having antibacterial properties. These properties are produced by incorporating a combination of raw materials, one of which is chitin fibre in a proportion of around 2% by weight. No biodegradable properties are claimed for this catheter, and it is unlikely that its rate of decomposition in the natural environment would be significantly increased.

### SUMMARY OF THE INVENTION

When viewed from one aspect the present invention proposes a biodegradable ISC (intermittent self catheterisation) catheter formed of a homogeneous blend of synthetic polymer and a natural biodegradable organic material.

In a preferred embodiment the natural biodegradable organic material is a fibrous substance. Such natural fibrous materials help to maintain the strength of the blended material whilst promoting breakdown in the environment by naturally occurring microorganisms.

Preferred examples include insoluble fibrous proteins such as collagen, which is inexpensive and can be extracted from animal skins and other waste animal products.

Other preferred examples of natural biodegradable fibrous materials are linear polysaccharides such as chitosan, which is derived from the shells of crustaceans such as shrimp, lobsters, and crabs. Chitosan is composed of linked acetylated and deacetylated glucosamine units.

In other embodiments the natural biodegradable organic material comprises polymeric carbohydrates, e.g. starches, which may be derived from cereal crops such as sorghum, millets, wheat, maize, and rice. Such natural polymeric carbohydrates can be directly consumed by microorganisms.

The natural biodegradable organic material preferably comprises at least 5% of the blended material, by weight, to exhibit the desired decomposition properties when exposed to the natural environment. The biodegradable organic material may comprise up to 35% of the blended material, by weight, without seriously degrading the mechanical properties of the catheter such as strength and flexibility.

The blend of materials used may incorporate functional additives which enhance the strength of the polymer. Such functional additives include cyclic amides such as caprolactam.

Other functional additives may include plasticisers, fillers and colourants, for example.

The functional additives may comprise up to 10% of the catheter material by weight.

The synthetic polymer may comprise any suitable catheter material, including at least one of low density polyethylene, high density polyethylene and polyvinyl chloride.

The invention also provides a catheter package comprising:
an envelope having walls formed of a material which is impermeable to gases;
an ISC (intermittent self catheterisation) catheter sealed within the envelope, wherein the catheter is formed of a blend of synthetic polymer and a natural biodegradable organic material.

### BRIEF DESCRIPTION OF THE DRAWINGS

The following description and the accompanying drawings referred to therein are included by way of non-limiting example in order to illustrate how the invention may be put into practice. In the drawings:

FIG. 1 is a general view of a biodegradable catheter as supplied in a catheter package prior to use;

FIG. 2 is a graph to illustrate the degradation of the catheter in the environment compared with a normal catheter.

### DETAILED DESCRIPTION OF THE DRAWINGS

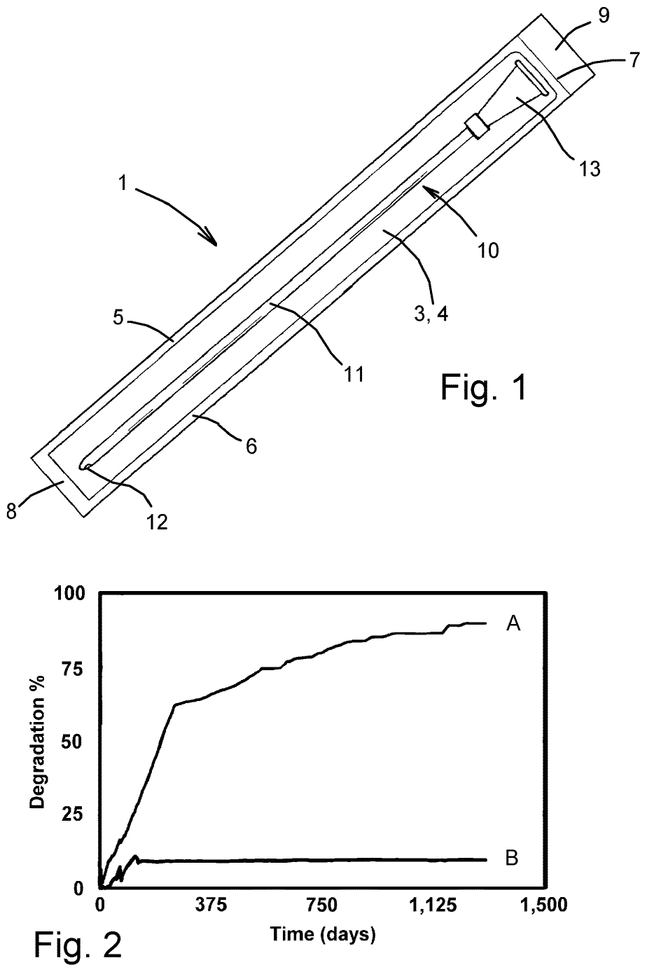

Referring firstly to FIG. 1, a package 1 includes a protective outer envelope 2 which contains a urinary catheter intended for intermittent self catheterisation of the bladder. The envelope is formed from front and rear sheets, 3 and 4, of flexible film material which is impermeable to gases and which provides a sterile environment for the catheter prior to use. The impermeable sheet or film material should be capable of excluding moisture in the form of liquid or water vapor for the recommended shelf life of the catheter package, which could be up to five years (typically 36 months). The rear sheet 4 may be opaque and printed with instructions and other identification material. The front sheet 3 may be of transparent sheet which an be vacuum formed to closely surround the contents of the envelope. The two sheets are welded together along their side edges 5 and 6 and along the top and bottom edges 7 and 8 to form an enclosed sterile and dry compartment. The sheets 3 and 4 from two flaps 9 at one end of the envelope by which the sheets can be peeled apart to open the package, breaking the top end seal 7.

The envelope 1 contains a urinary catheter 10 which is enclosed by the envelope. The catheter 10 has a catheter tube 11 dimensioned for introduction through the urethra, with a distal end provided with one or more urine inlet openings 12 and an opposite proximal end provided with an outlet connector 13 by which the catheter may be joined to a flexible pipe for conducting urine withdrawn from the bladder to a urine collection bag (not shown).

The package is manufactured under dry and sterile conditions so that the catheter 10 is protected by the outer envelope 2 against contamination by moisture, dirt or bacteria.

The catheter 10 (tube 11 and connector 13) is formed from a biodegradable plastic which comprises a homogeneous blend of synthetic polymer and a natural biodegradable organic material, three examples of which are given below.

### EXAMPLE 1

The biodegradable plastic comprises a blend of the following components by percentage weight:
polyvinyl chloride 60%
collagen 30%
caprolactam 3%
plasticisers, fillers, colourants etc. 7%

### EXAMPLE 2

The biodegradable plastic comprises a blend of the following components by percentage weight:
low density polyethylene 70%
chitosan 25%
plasticisers, fillers, colourants etc. 5%

### EXAMPLE 3

The biodegradable plastic comprises a blend of the following components by percentage weight:
polyvinyl chloride 75%
starch 20%
plasticisers, fillers, colourants etc. 5%

In each example the materials are kneaded and blended at 130 to 140° C. to form a homogeneous mix which is then sent to a screw-extruder and pelletised at around 150° C. The pelletised biodegradable plastic is then used to mould the catheter, which is sealed into the envelope under sterile conditions.

After storage for a period of up to five years in the sealed envelope, the catheter is suitable for use in intermittent self catheterisation without degradation.

As shown in graph A of FIG. 2, the addition of the biodegradable natural organic material significantly increases the speed of decomposition of the catheter when exposed to the natural environment, e.g. landfill, as compared with a catheter formed of normal non-biodegradable polymer—graph B. The natural organic material attracts microbes to the surface of the article where they excrete enzymes. The material also acts microscopically to provide a greater surface area for the microbes to attach to the catheter. These enzymes interact with the blend of polymer and organic material to soften the polymer bonds that allow the microbes to digest the catheter. As a result, the catheter is 90% degraded after a period of about 4 years compared with less than 10% degradation of a normal catheter. Furthermore, the present catheter continues to decompose whereas the normal catheter exhibits no further degradation.

It is important that any colourants used are ultra-low toxicity, minimising the presence of toxic elements such as zinc, copper, mercury, chromium etc. so as not to cause a negative impact on the environment and normal microbial activity.

Depending on the thickness of the catheter and the materials used, adding the organic material to the synthetic polymer may be sufficient to cause the catheter to degrade by almost 100% in less than 4 years. Without the added organic material the catheter may exhibit little or no degradation over the same period. The catheter can be broken down 200 times faster, leaving only water, biomass, and carbon dioxide and methane, molecules that naturally occur during biodegradation of all organic materials. Degradation has been shown to occur in anaerobic (without oxygen) and aerobic (with oxygen) environments, which means biodegradation will take place in equally well in landfill, compost and marine environments. Unlike chemically degradable plastics the degradation process does not leave any microplastics behind. In landfill environments methane is also released which can be collected and used to provide energy.

The catheters are therefore suitable to be disposed of in regular collection schemes, but when sealed within the moisture-excluding envelope the catheters still have a normal shelf life.

Whilst the above description places emphasis on the areas which are believed to be new and addresses specific problems which have been identified, it is intended that the features disclosed herein may be used in any combination which is capable of providing a new and useful advance in the art.

1. A biodegradable ISC (intermittent self catheterisation) catheter formed of a homogeneous blend of synthetic polymer and at least 5% by weight of a natural biodegradable organic material.

2. A catheter according to claim 1 wherein the natural biodegradable organic material is a fibrous substance.

3. A catheter according to claim 2 wherein the natural biodegradable organic material comprises an insoluble fibrous protein.

4. A catheter according to claim 3 wherein the insoluble fibrous protein comprises collagen.

5. A catheter according to claim 1 wherein the natural biodegradable organic material comprises a linear polysaccharide.

6. A catheter according to claim 5 wherein the linear polysaccharide comprises chitosan.

7. A catheter according to claim 1 wherein the natural biodegradable organic material comprises a polymeric carbohydrate.

8. A catheter according to claim 7 wherein the polymeric carbohydrate is a starch.

9. A catheter according to claim 8 wherein the polymeric carbohydrate is derived from a cereal crop.

10. A catheter according to claim 1 wherein the natural biodegradable organic material comprises less than 35% of the blended material by weight.

11. A catheter according to claim 1 which incorporates a functional additive.

12. A catheter according to claim 11 wherein the functional additive comprises a cyclic amide.

13. A catheter according to claim 12 wherein the cyclic amide is caprolactam.

14. A catheter according to claim 11 wherein the functional additive comprises one or more of plasticisers, fillers and colourants.

15. A catheter according to claim 11 wherein the functional additive comprises from 1% to 10% of the catheter material by weight.

16. A catheter according to claim 1 wherein the synthetic polymer comprises at least one of low density polyethylene, high density polyethylene and polyvinyl chloride.

17. A catheter package comprising:
an envelope which is impermeable to gases;
an ISC (intermittent self catheterisation) catheter according to claim 1 which is sealed within the envelope.
